(19) United States
(12) Patent Application Publication
(10) Pub. No.: US 2025/0152375 A1
(43) Pub. Date: May 15, 2025

(54) PIVOTING SHOULDER IMPLANT TRAY

(71) Applicant: Zimmer, Inc., Warsaw, IN (US)

(72) Inventor: Jai Abhishekh Veezhinathan, Warsaw, IN (US)

(21) Appl. No.: 18/940,491

(22) Filed: Nov. 7, 2024

Related U.S. Application Data
(60) Provisional application No. 63/597,591, filed on Nov. 9, 2023.

(51) Int. Cl. A61F 2/40; A61F 2/30

(52) U.S. Cl. CPC A61F 2/4014; A61F 2/30749; A61F 2002/30649; A61F 2002/4022; A61F 2002/4085

(57) ABSTRACT

An implantable device configured to have at least one articulation component. The implantable device including a tray member configured to be attached to a receiving bone. The tray member includes at least first side configured to face the receiving bone, a second side opposite to the first side and including a cavity opening and a cavity defined within the tray member and accessible through the cavity opening. The implantable device includes an articulation member an articular surface where the articulation member is configured to be operably coupled to the tray member such that the articulation member can move within the cavity.

### CLAIM OF PRIORITY

This application claims the benefit of U.S. Provisional Patent Application Ser. No. 63/597,591, filed on Nov. 9, 2023, the benefit of priority of which is claimed hereby, and which is incorporated by reference herein in its entirety.

### TECHNICAL FIELD

Examples described in this document pertain generally, but not by way of limitation, to an orthopedic system. More specifically, examples herein generally relate to and describe a component of a joint replacement implant system, such as a shoulder implant system.

### BACKGROUND

In a healthy shoulder, the proximal humerus is generally ball-shaped and articulates within a socket formed by a lateral portion of the scapula, called the glenoid, to form the shoulder joint. Some implant systems for a total replacement of the shoulder joint can generally replicate the natural anatomy and movement of the shoulder. Such implant systems can include a humeral component having a stem that fits within the humeral canal and an articulation head that articulates within the socket of a glenoid component implanted within the glenoid of the scapula. Reverse-type shoulder implant systems have been developed in which the conventional ball-and-socket configuration, which replicates the natural anatomy of the shoulder, is reversed. For example, a reverse-type shoulder implant system can include a concave recessed articulation component at the proximal end of the humeral component, which can articulate against a convex portion of a glenoid component.

The design of humeral implants for shoulder arthroplasty has evolved over the years. Whether standard or reverse, the humeral component of traditional shoulder arthroplasty is traditionally designed with a stemmed implant. The humeral component can include a humeral stem that statically holds a humeral cup. This can allow the glenoid sphere component of the implant system to articulate within the humeral cup. However, current designs of humeral cups do not allow for full articulation similar to that of a healthy shoulder joint. In current designs of a humeral cup (or reverse design), the glenoid sphere portion can move a limited amount, as dictated by the design of the humeral cup.

### SUMMARY

The present inventors have recognized, among other things, one problem to be solved can include increasing the articulation of a shoulder implant system. This can include, for example, increasing the degrees of the movement of a glenoid sphere component in cooperation with a humeral tray (in traditional or reverse shoulder arthroplasty). The present subject matter can help provide a solution to this problem, such as with an implanted tray that can articulate relative to a supporting stem.

In examples, an implantable device can include a tray member that can be attached or coupled to a receiving bone. The tray member can include a first side configured to face the receiving bone, a second side opposite to the first side and a cavity with a cavity opening facing away from the first side. The cavity can be accessible through the cavity opening.

In an example, an articulation member can be configured to be at least partially retained within the cavity. The articulation member can include, for example, an articular surface configured to face away from the cavity and a base surface that can face towards the cavity. The articulation member can be operably coupled to the tray member such that the articulation member can pivot within the cavity.

In examples, an articulation implant can include a static tray member that can be coupled with a receiving bone. The static tray member can include a first side configured to face the receiving bone, a second side opposite to the first side and having a cavity opening, and an inner wall of the tray member defining a cavity that is accessible through the cavity opening. The articulation implant can also include an articulation member configured to be at least partially retained within the cavity. The articulation member can include an articular surface configured to face away from the cavity, and a base surface configured to face toward the cavity. The articulation implant can also include a bearing configured to articulate the articulation member relative to the tray member.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is an isometric view a surgical implant in accordance with at least one example of the present disclosure.

FIG. 1B is cross section of a surgical implant in accordance with at least one example of the present disclosure.

FIG. 1C is a top view of a surgical implant in accordance with at least one example of the present disclosure.

FIG. 2 is a cross section view of a tray component of a surgical implant in accordance with at least one example of the present disclosure.

FIG. 3 is a cross section view of an articulation component of a surgical implant in accordance with at least one example of the present disclosure.

FIG. 4 is a cross section view of a tray component of a surgical implant in accordance with at least one example of the present disclosure.

FIG. 5A-D are cross section views of a surgical implant system in accordance with at least one example of the present disclosure.

FIG. 6 is an isometric view of a bearing in a surgical implant in accordance with at least one example of the present disclosure.

FIG. 7 is a cross section view of a surgical implant in accordance with at least one example of the present disclosure.

FIGS. 8A-8C are views of movement of a surgical implant in accordance with at least one example of the present disclosure.

FIG. 9 is a cross section view of a surgical implant in accordance with at least one example of the present disclosure.

### DETAILED DESCRIPTION

As used herein, the following directional definitions apply. Anterior and posterior mean nearer the front or nearer the rear of the body, respectively, proximal, and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

A healthy shoulder joint includes a glenohumeral joint. The glenohumeral joint is a ball and socket joint that allows articulation of the scapula and the proximal humerus. The head of the humerus contacts the glenoid cavity of the scapula such that the head of the humerus is able to articulate, move, pivot or the like within the glenoid cavity. Due to the anatomical structure of the glenoid cavity and the size of the head of the humerus, the shoulder joint is one of the most mobile joints in the human body. Because of the increased mobility of the shoulder joint, the shoulder joint can become dislocated or injured easily. In some instances, shoulder surgery, including a shoulder joint replacement, can be necessary to repair the injured joint. While a shoulder joint is discussed throughout, the concepts disclosed herein can be applied to any joint replacement which utilize at least two interacting components, such as one having a curved or round surface and the other having a receiving or cooperating surface onto which the curved or rounded surface articulates or moves against.

In instances where a shoulder joint replacement may be necessary, an implant system including at least a glenoid sphere and a humeral insert can be implanted into the existing bones, namely the humerus and the scapula. For example, a humeral stem component can be inserted into the humerus bone. The humeral stem can have a medially facing humeral head. The humeral head can be formed to mimic the anatomical profile of healthy articular cartilage at the superior end of the humerus. In an example, the humeral head can contact a glenoid component implanted in the scapula. The glenoid component can have a surface on which the humeral head is able to articulate. In other examples, the system can be reversed and at the superior end of the humeral stem (implanted in the humerus) is a humeral cup having a surface against which a glenosphere (implanted in the scapula) articulates.

In traditional shoulder implant systems, the surface, such as a tray, onto which a rounded component moves, can be statically implanted, similar to the anatomical profile and structure of a healthy shoulder joint or other similar ball and socket joint. However, because the tray is not the anatomical structure, there can be limited mobility. While this description is directed to a shoulder joint, the concepts in this description can relate to any ball and socket joint, or other similar joint.

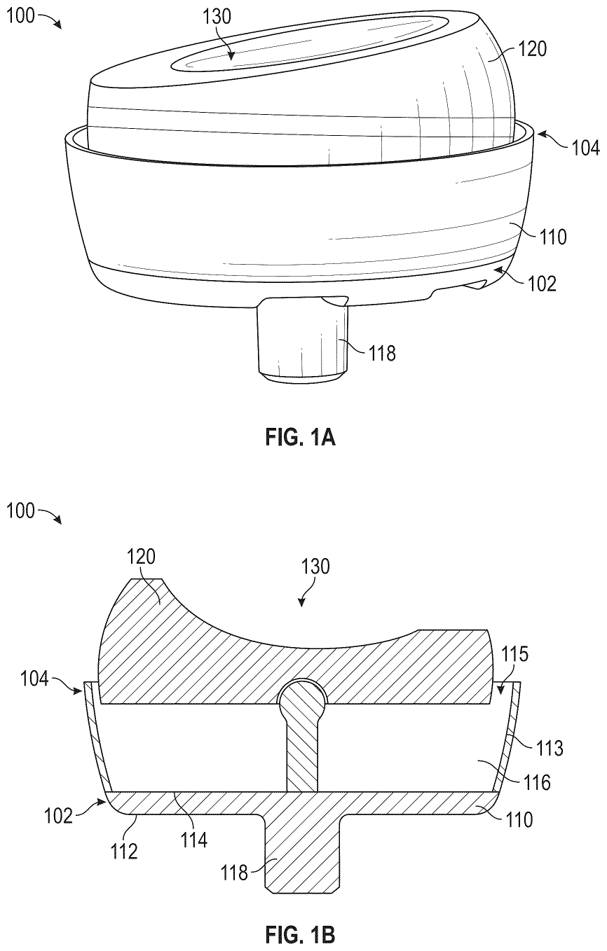

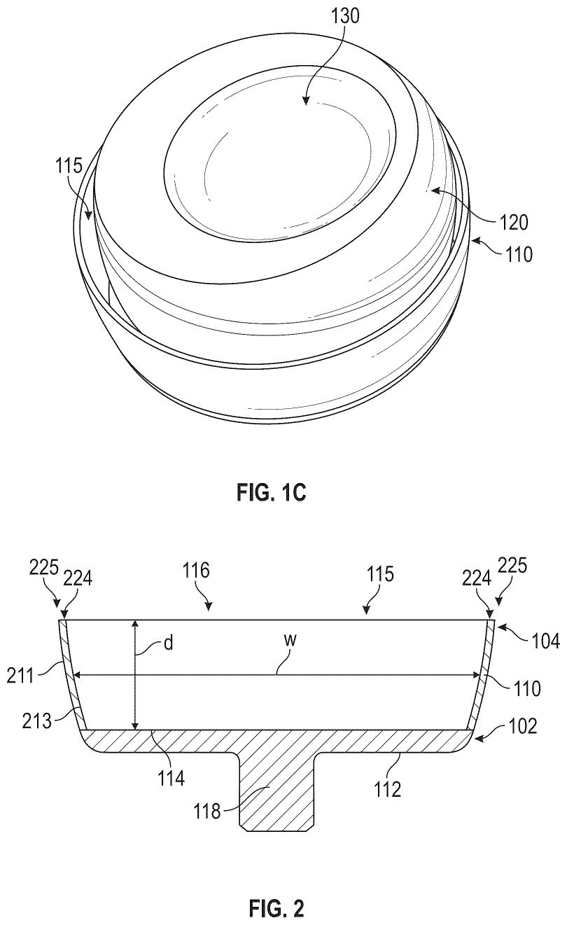

FIGS. 1A and 1B illustrate an example of an implantable device 100 that can increase mobility of the joint after implantation surgery. The implantable device 100 can include at least two components. One component of an implant system that can be used in replacement of a socket portion of a joint includes, for example, a concave surface that can move in response to applied forces. The other component can be static and at least partially implanted in a receiving bone. In an example, the implantable device 100 can replace the glenoid cavity. The implantable device 100 can be inserted in either the humerus or the scapula, as dictated by the implant surgery.

Illustrated in FIGS. 1A-1C is an example two-part implantable device 100 including a tray member 110, as the static component that can be implanted into a receiving bone, and an articulation member 120, as the movable portion. The articulation member 120 can have a concave profile 130 that can receive a complementary implant that mimics a corresponding portion of a joint. The tray member 110 can extend from a proximal portion 102 that can be proximate to or in contact with a receiving bone and to a distal portion 104 that can be a specified distance removed from the proximal portion 102. In an example, a stem 118 can extend from the proximal portion 102 of the tray member 110. The stem 118 can be formed for implantation into a receiving bone.

As illustrated in FIG. 1B, the proximal portion 102 can include a first, outer, side 112 and a second, inner, side 114. For instance, the first, outer, side 112 can be an outer wall that contacts or is proximate to the receiving bone into which the implant can be inserted and the second, inner, side 114 can be an inner wall that is the base portion of a cavity 116 within the tray member 110.

An outer wall 113 can surround the cavity 116. In an example the outer wall 113 can extend toward the tray member 110 from the distal portion 104 such as from a cavity opening 115, to the second, inner, side 114. In an example the outer wall 113 can be bowl-shaped such as a circumference of the outer wall 113 proximate to the cavity opening 115 is wider than the portion of the outer wall 113 proximate to the tray member 110.

In an example shown in FIG. 1C, the cavity opening 115 of the tray member 110 can receive or retain the articulation member 120. The articulation member can 120 be centered within the tray member 110, or disposed closer to a side of the tray member 110. The articulation member 120 can articulate, pivot, rotate or otherwise move in a controlled manner within the distal portion 104 of the tray member 110, such as within the cavity opening 115.

Illustrated in FIG. 2 is an example of a cross section of the tray member 110. The tray member 110 can have a peripheral outer wall 211, as side portions, the first side 112, as the base, and an inner wall 213 that, along with the second side 114, can define the confines of the cavity 116. The cavity 116 can be accessible through the cavity opening 115 and can extend a specified depth within the tray member 110 terminating at the second side 114 (inner base surface). The cavity opening 115 can be defined by a rim 225 extending around or encircling distal the perimeter of tray member 110.

The depth d of the cavity 116 can extend from the rim 225 at the distal portion 104 or the cavity opening 115 to the second side 114 at the proximal portion 102. The width w, such as a diameter, of the cavity 116 can be dictated by the size of the articulation member 120 associated with the tray member 110. The dimensions of the cavity 116 can be sized to allow for movement or articulation of the articulation member 120.

The second side 114 can be a planar or flat surface at the base of the cavity 116. In another example the second side 114 can be a concave or a convex surface at the base of the cavity 116. The peripheral outer wall 211 and the inner wall 213 can be convex or concave surfaces that meet with the first side 112 and second side 114, respectively.

On the first side 112 of the tray member 110, or the outer wall opposing the second side 114, can be the stem 118 as the attachment component of the tray member 110 that can be implanted in the receiving bone. For example, the stem 118 can extend, protrude or be coupled to the first side 112 of the tray member 110. The stem 118 can be an example component of the implantable device 100 that is designed to be inserted into a receiving bone such as the humerus or the scapula. The stem 118 can be sized and shaped to be statically implanted and retained within the bone. In an example, the length of the stem 118 can be shorter when the implantable device 100 is implanted in the scapula than when the stem 118 is implanted in the humerus. The width, such as a diameter, of the stem can be dependent on the dimensions of the tray member 110. For example, for a larger tray member 110 the stem 118 can be wider than for a smaller tray member 110. In other examples, the tray member 110 can be a “stemless” implant and coupled with the receiving bone according to known methods.

The distal portion 104 of the tray member 110 can include for example, a rotation stop 224 proximate to, or at, a rim 225 of the tray member 110. For example, the rim 225 can be a surface of the tray member 110 that defines a perimeter of the cavity opening 115. The rim 225 can be the outer perimeter of the tray member 110 most remote from the bone. For example, the rotation stop 224 can be a surface or similar feature that prevents or inhibits movement of the articulation member 120 such as over-rotation or over-articulation. In one example, the rotation stop 224 can be a feature added to the rim 225 of the tray member 110. In another example, the rotation stop 224 can be formed as a part of the rim 225 of the tray member 110, such as a lip or other protrusion or extension.

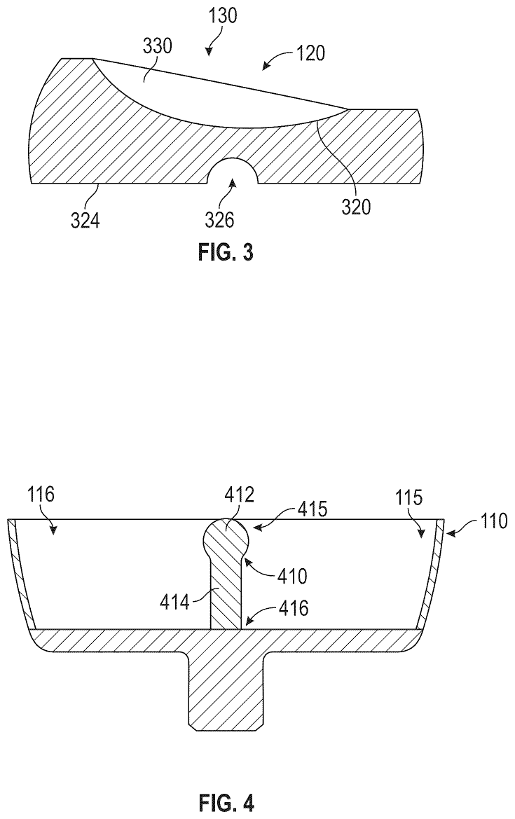

Illustrated in FIG. 3 is an example of the articulation member 120. The articulation member 120 can be a movable component of the implantable device 100. For example, the articulation member 120 can be a pivotable or rotatable part of the implantable device 100. The articulation member 120 can be designed to pivot, rotate, articulate or otherwise move with respect to the tray member 110. The articulation member 120 can have an articular surface 320 and a base surface 324. The articular surface 320 can face away from the receiving bone into which the tray member 110 has been implanted or away from the cavity 116. The base surface 324 can face the receiving bone into which the tray member 110 has been implanted or towards the cavity 116.

The articular surface 320 can be formed to engage with or receive an opposing component, a complementary implant, of an implant system for a ball and socket or similar joint. In an example, the articular surface 320 can engage with or receive a glenoid sphere or similar rounded complementary implant. In an example, the articular surface 320 can be a planar surface, a concave surface or a convex surface. In an example the articular surface 320 can include a crater, recess, pocket, depression, or other concave form within the articular surface 320. The profile of the articular surface 320 can be designed to be a corresponding receiving surface for the complementary implant of the implant system, such as a rounded, component to replicate a ball of a ball joint. For example, the concave profile 130 can be centered within the articular surface 320, such as a symmetric articular surface 320. In another example, the articular surface 320 can be asymmetric such that one of the articular surface slopes at a larger angle from one side of the articular surface 320 to another side.

In an example, the articulation member 120 can have a liner 330 coupled to the articular surface 320. In an example, the liner 330 can have a profile similar to the articular surface 320. In another example, the liner 330 can have a different profile than the articular surface 320. The liner 330 can provide a smoother, or less resistive, surface when the implantable device 100 is associated with, for example, a glenoid sphere. In an example, the liner 330 can be compatible with an opposing glenoid sphere, or similar rounded surface. The liner 330 can be formed from a polymer material, metallic material, or any other suitable material that can provide a relatively smooth surface.

On the opposing side of the articulation member 120 from the articular surface 320 can be the base surface 324. The base surface 324 can be a planar, flat, concave, convex or any combination of surface profiles suitable for movement of the articulation member 120. In an example the base surface 324 can have a recess 326, such as a groove, an indentation, or the like. The recess 326 can be located at any position in the base surface 324 that is suitable for receiving a bearing engagement, as will be discussed below. For example, the recess 326 can be centrally located in the base surface 324. The recess 326 can be a pivot point of the articulation member 120.

Illustrated in FIG. 4 is an example tray member 110 with a bearing 410 that can extend or protrude from the second side 114. The bearing 410 can be a ball bearing 412 located at the end of shaft 414, such as a screw, a bolt, a peg, or the like. In one example, a roller ball bearing can be provided at a distal end 415 of the shaft 414. In another example, the bearing 410 can be static and can have a rounded profile that enables another structure, such as the articulation member 120, to rotate, pivot or otherwise move. The shaft 414 can be an example of a support structure that can extend, protrude or otherwise be coupled with the second side 114. For example, the shaft 414 can be a separately formed component coupled within the cavity 116. The shaft 414 can be coupled at a proximal end 416 to the tray member 110 and can extend towards the cavity opening 115 with the ball bearing 412 supported at a distal end of the shaft 414.

The bearing 410 can be centrally located within the cavity 116 or at any location within the cavity 116 that allows for the articulation member 120 to rotate, pivot or otherwise move.

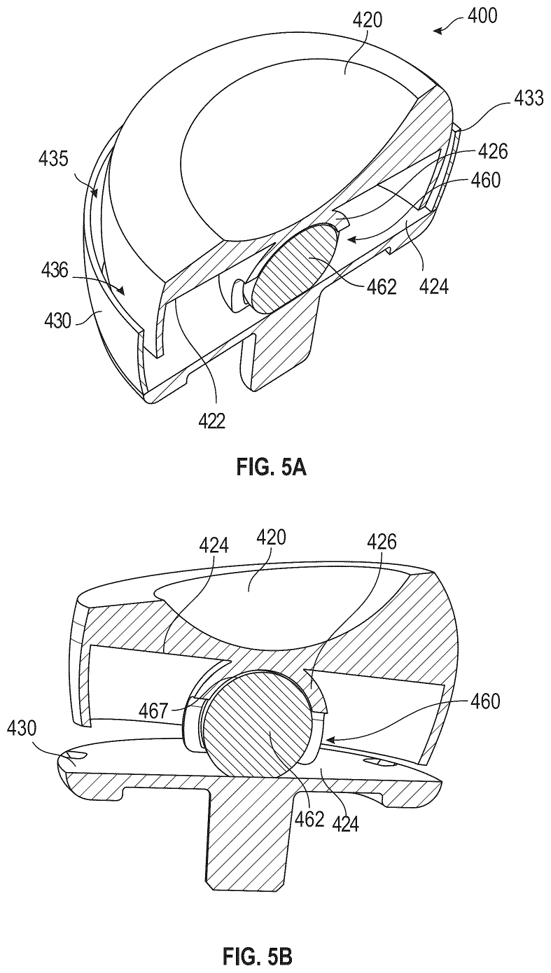

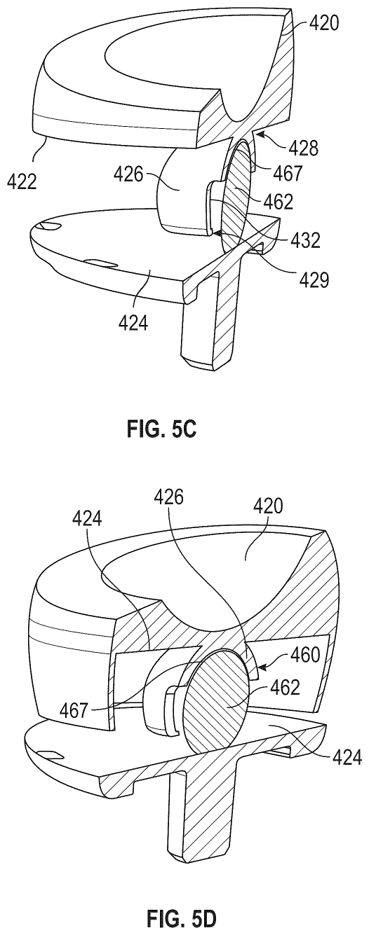

FIGS. 5A-5D illustrate examples of a design of a bearing assembly 460 of an implantable device 400 including outer surfaces and profiles similar to the implantable device 100 illustrated in FIGS. 1A-4. The implantable device 400 can include an articulation member 420 and an associated tray member 430. The articulation member 420 illustrated in FIGS. 5A-5D can have a similar profile to the articulation member 120 illustrated in FIGS. 1A-4. The tray member 430 can be similar to the tray member 110 illustrated in FIGS. 1A-4.

The tray member 430 optionally includes one or more outer walls 433 extending around a cavity 436. The one or more outer walls 433 can also define an opening 435 sized and shaped to receive the articulation member 420. The cavity 436 extends from an opening 435 to a base surface 424 of the tray member 430. The opening 435 and the cavity 436 can be sized to allow the articulation member 420 to articulate, pivot, move or the like within the cavity 436 of the tray member 430. The articulation member 420 can be positioned within a tray member 430. For example, the articulation member 420 can be positioned within a cavity 436 of the tray member 430.

The articulation member 420 can include a bearing assembly 460. The bearing assembly 460 includes a bearing 462 extending or protruding from a base surface 424 of the tray member 430. The bearing 462 is formed to fit into a cage 426. The cage 426 extends or protrudes from an inner facing surface 422 of the articulation member 420. In an example, the cage 426 is molded as a singular component from the inner facing surface 422 of the articulation member 420. In an example, the cooperation between the cage 426 and the bearing 462 can allow the articulation member 420 to move, pivot, articulate or the like in a range of angles and orientations.

As illustrated in FIGS. 5A-5D, the cage 426 can protrude or extend from the inner facing surface 422 having a concave profile, a recess, indent, or the like, and open towards a cavity 436 within the tray member 110. The cage 426 can be semi-spherical, rounded, or any other shape that can receive the corresponding bearing 462. The cage 426 can be positioned to be centered on the inner facing surface 422. In another example, the cage 426 can be positioned to be off-center. The cage 426 can extend towards the base surface 424 an equal distance around the periphery of the cage 426. The cage 426 can vary dimension, such as in length from a proximal portion 428 of the cage 426 to the distal portion 429 of the cage 426. The cage 426 can also vary in width or diameter according to a specified purpose and design of the articulation member 420. The cage 426 can further include at least one window or opening 432 disposed along specified portions of the periphery of the cage 426. The opening 432 can have any size or shape that can allow for ease in coupling the bearing 462 with the cage 426. The opening 432 can allow for an increased in flexibility of the movement of the articulation member 420. The opening 432 can also provide for increased tolerances for stresses the cage 426 can accommodate or withstand.

The bearing 462 illustrated in FIGS. 5A-5D can be coupled to or extend from base surface 424 of the tray member 430. The bearing 462 can extend away from the base surface 424. In an example, the bearing 462 can be spherical, ellipsoid, hemispherical or the like. The bearing 462 can be any shape or profile that couples with the cage 426. The bearing 462 can have a size and profile that retained within the cage 426. In an example, the cage 426 is retained by the bearing 462 to pivot, articulate or otherwise more relative to the bearing 462. For example, the bearing 462 can have a size and profile that temporarily contacts the inner surface 467 of the cage 426 during movement such as pivoting, articulation or the like.

FIG. 6 is an example of perspective view of the bearing assembly 460 with the opening 432. The opening 432 can allow for a more fluid movement of the cage 426 about the bearing 462. The bearing 462 in FIG. 6 fits within the cage 426 to allow the articulation member 420 to pivot, rotate, articulate, or otherwise move relative to the base surface 424 of the tray member 430.

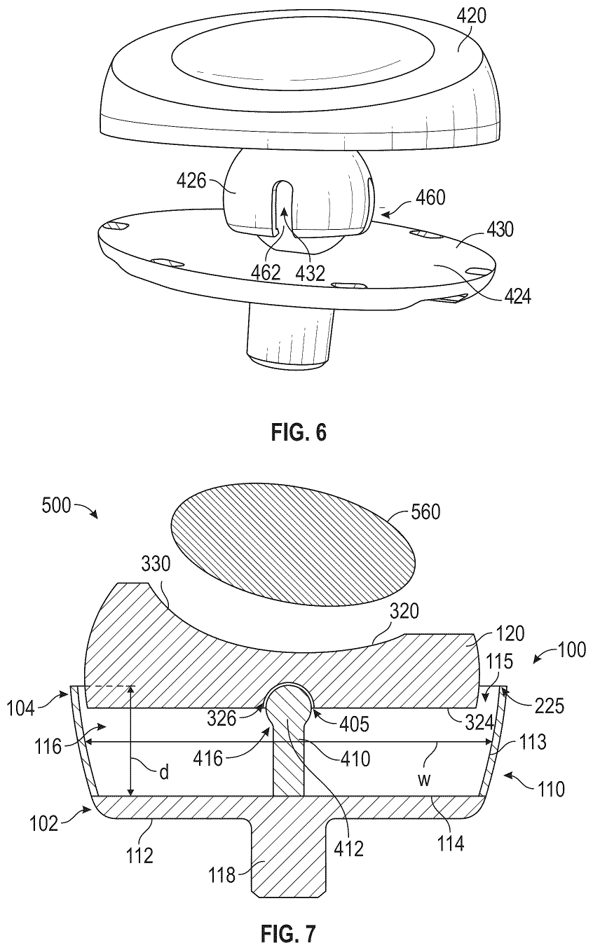

Illustrated in FIG. 7 is an example of an implant system 500 including a bearing assembly 405 and a complementary implant 560. The implant system 500 can include the implantable device 100 as discussed related to FIG. 1A or 1B. The implantable device 100 can include an assembly of the example components illustrated in FIGS. 2, 3 and 4. For example, the implantable device 100 can include the tray member 110 (while tray member 110 is illustrated, any tray member previously described can be incorporated). The tray member 110 can include at least one of a cavity 116, an articulation member 120 and a bearing 410 within the cavity 116. The tray member 110 can include one or more walls 113 that extend around the cavity 116. The cavity 116 can extend from the cavity opening 115 to the second side 114 (e.g., a base surface) of the tray member 110. The depth d and the width w of the cavity 116 can be sized to allow the articulation member 120 to rotate, pivot, move or otherwise articulate. As discussed, related to FIG. 2, an upper portion of the one or more walls 113 surrounds the cavity opening 115. The upper portion of the one or more walls 113 and include a rim 225. The rim 225 can include a protrusion or extension positioned over or extending toward the cavity 116. The rim 225 can be one element of the implantable device 100 that allows for controlled movement of the articulation member 120.

Similar to the articulation member 120 discussed related to FIG. 3, the articulation member 120 can have an articular surface 320 and a base surface 324. The articular surface 320 can be an engaging or receiving surface for a complementary implant 560 that can be a component part of the implant system 500. The complementary implant 560 can have a form or profile that can slide, pivot, translate, articulate, or otherwise move within or on the articular surface 320. In one example, the complementary implant 560 can have a rounded profile. The complementary implant 560 in cooperation with the implantable device 100 can provide a specific range of motion of a newly formed or repaired joint. The articular surface 320 can react or transmit to forces originating from movement of and engagement with a rounded portion of a complementary implant 560. For example, the articulation member 120 can pivot, rotate, translate, articulate, or otherwise move in relation to the movement of the complementary implant 560. The additional movement of the articulation member 120 can generate a wider range of motion or articulation in a joint with the implant system 500.

The articulation member 120 can be retained within the distal portion 104 of the tray member 110. In an example, the articulation member 120 can be snap fit, inserted, or otherwise retained in the distal portion 104 while still allowing the articulation member 120 to move within the cavity opening 115. For example, the articulation member 120 can be partially retained within the cavity 116 with the base surface 324 of the articulation member 120 extending into the cavity 116 and an articular surface 320 extending distally away from the cavity 116. In another example, the outer surface, or articular surface 320, can be positioned below the rim 225, partially below or even above the rim 225.

In one example, the rim 225 can assist in retention of the articulation member 120 with the articular surface 320. For example, the rim 225 assist in inhibiting over-articulation, over-pivoting or over-rotation of the articulation member 120 by acting as a brace.

The complementary implant 560 can be retained within a concave articular surface 320. For example, the articular surface 320 can be concave and have a focal point centrally located on the articular surface 320 about which the complementary implant 560 pivots, rotates or otherwise moves. In another example, the focal point can be located a specified distance spaced from the center.

The articular surface 320 can be a substantially smooth surface to minimize friction during movement of a complementary implant portion that engages with the concave articular surface 320. For example, the concave articular surface 320 can include a liner 330 formed from a material that has a low coefficient of friction. The liner 330 can be coupled with the articular surface 320 according to methods known in the art.

The base surface 324 can face into the cavity 116. In an example, the base surface 324 can be substantially planar or flat. The base surface 324 can also have a recesses, indentations, protrusions or forms extending from or extending into the base surface 324 into the cavity 116. For example, the base surface 324 can have a recess 326. The recess 326 can be, in one example, centrally located in the base surface 324. The recess 326, in another example, can be located off-center. The recess 326 can have a suitable profile that allows the articulation member 120 to pivot, rotate, articulate or otherwise move within the tray member 110.

The recess 326 working in combination with the bearing 410 can be a bearing assembly 405. The recess 326 can be sized and/or shaped to receive a portion of the bearing 410. The bearing assembly 405 can provides cooperation between the articulation member 120 and the tray member 110. The bearing assembly 405 of the bearing 410 in combination with the recess 326 can be an example of a component that controls movement of the articulation member 120 in response to forces translated from a complementary implant component during articulation of the joint.

The interaction between the bearing 410 and the recess 326 of the articulation member 120 can cause the articulation member 120 to be movably coupled within the tray member 110. The articulation member 120 can be movably coupled with the tray member 110 due to the bearing 410 providing a pivot or articulation point.

The assembled implantable device 100, including the articulation member 120 and the tray member 110 can be implanted into a receiving bone. The tray member 110, for example, includes a stem 118 that extends from the first side 112 (e.g., outer base surface). The stem 118, for example, can be implanted or inserted into a receiving bone, such as the humeral bone. In an example, the complementary implant 560 can be inserted into a complementary part of a shoulder girdle, such as a clavicle.

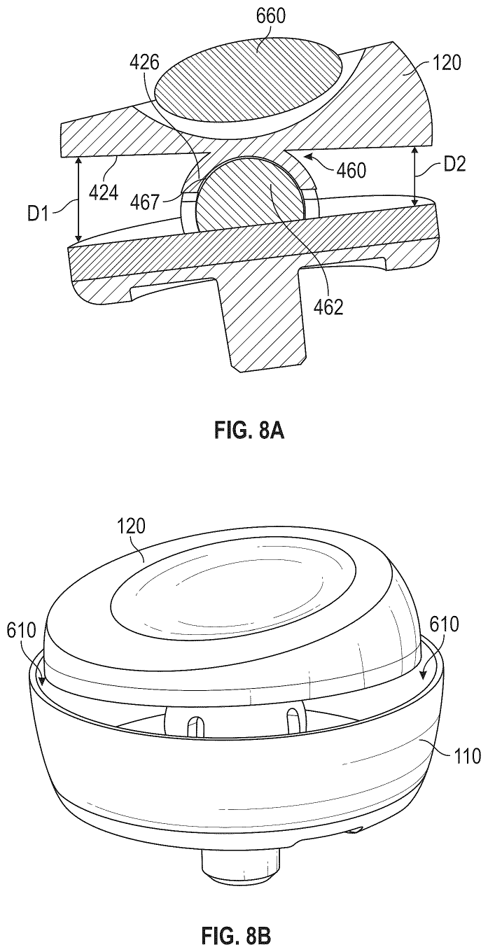

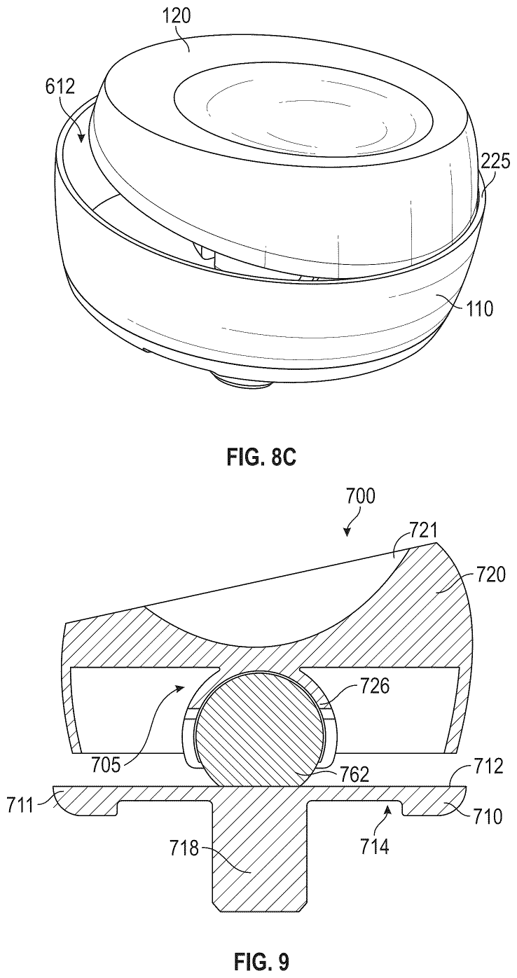

In the examples shown in FIGS. 8A-8C the movement of the articulation member can result from forces translated to a bearing assembly 460 or bearing assembly 405 (as illustrated in FIG. 4 or 7). In FIG. 8A, the articulation member 120 is illustrated in an engaged position where a complementary implant 660 can apply pressure or a force against the articulation member 120. As illustrated in FIG. 8A, the distance D2 between the side receiving the force can be less than the distance D1 of side not receiving the force. In this example, the cage 426 pivots, rotates, articulates or otherwise moves against the bearing 462.

Illustrated in FIGS. 8B and 8C are perspective views of movement of the articulation member 120. As illustrated in FIG. 8B, the articulation member 120 can be substantially centered within the tray member 110. For example, the space 610 is substantially equal around the perimeter of the articulation member 120. In another example, the articulation member 120 can have substantially equal spaces 610 on opposing sides of the articulation member 120. For example, the space 610 between the articulation member 120 and the tray member 110 is similar (e.g., equal, within less than a millimeter, within less than 5 millimeters). The articulation member 120 of FIG. 8B illustrates an example with minimal force is applied to the articulation member 120. In the position with force is not, or minimally, applied to the articulation member 120, the articulation member 120 can be in a resting position or orientation.

Illustrated in FIG. 8C, is an example of the articulation member 120 with a force applied to a least a portion of the articulation member 120. The articulation member 120 can be angled or aligned towards one side of the tray member 110 when a force is applied. In this example, the articulation member 120 can have a force applied to it from the complementary implant, such as in the illustration in 8A. In the example illustrated in FIG. 8C, a space 612 can be formed between the articulation member 120 and the tray member 110 that is greater than the space between an opposing side of the articulation member 120 and the tray member 110. In this example, the articulation member 120 is limited in its movement by the rim 225 of the tray member 110. The articulation member 120 can pivot, rotate, articulate or otherwise move within the limitations of the rim 225 of the tray member 110.

Illustrated in FIG. 9 is another example of an implantable device 700. The implantable device 700 is similar to the implantable device 100, as discussed related to FIG. 1A or 1B. The implantable device 700 can include an articulation member 720, similar to the articulation member 120 or the articulation member 420 discussed previously. The implantable device 700 can receive an implant within an articular surface 721. The articular surface 721, for example, receives and transmits forces applied to the articulation member 720 in response to movement of a joint into which the implantable device 700 is implanted.

The implantable device 700 can include a tray member 710 having a base 711 with a stem 718 extending in a direction away from a first side 712 of the tray member 710. The stem 718 can be formed to be inserted into a bone. The base 711 can be a planar surface with a first side 712 and a second side 714. The first side 712 of the base 711 can pivotally support the articulation member 720 and the second side 714 can be formed to be positioned against a bone.

In an example, the implantable device 700 can include a bearing assembly 705 similar to the bearing assembly 460 or bearing assembly 405 discussed previously. The bearing assembly 705 can include a bearing 762 coupled with the tray member 710 and a cage 726 coupled with the articulation member 720. The cage 726 can be formed to move, pivot, articulate or the like about the bearing 762. The movement of the cage 726 relative to the bearing 762 can provide added range of motion to the implantable device 700.

The bearing assembly 705 can control pivoting movement of the articulation member 720 relative to the base 711. For example, the articulation member 720 can pivot within the limitations of the bearing assembly 705. For example, the articulation member 720 can pivot or articulate in any direction according to the movement of the joint and is not limited by a peripheral outer wall as discussed previously related to FIG. 2. The articulation member 720 can be limited in movement by an interaction between the articulation member 720 and the first side 712 of the tray member 710 instead of a rim and wall, such as discussed in previous Figures. In some examples, the implantable device 700 allows for a wider range of movement of the joint as compared to the implantable device discussed previously a tray member that includes a wall surrounding a cavity.

### VARIOUS NOTES AND ASPECTS

Aspect 1 can include an implantable device comprising a tray member configured to be attached to a receiving bone, the tray member including a first side configured to face the receiving bone, a second side opposite to the first side and including a cavity opening, a cavity defined within the tray member and accessible through the cavity opening; and an articulation member configured to be at least partially retained within the cavity, the articulation member including an articular surface configured to face away from the cavity and a base surface configured to face towards the cavity; wherein the articulation member is configured to be operably coupled to the tray member such that the articulation member can pivot within the cavity.

Aspect 2 can include, or can optionally be combined with the subject matter of Aspect 1, to optionally include a liner coupled to the articular surface where the liner comprises a concave surface or convex surface.

Aspect 3 can include, or can optionally be combined with the subject matter of one or any combination of Aspects 1 or 2 to optionally include the articular surface comprises a concave surface.

Aspect 4 can include, or can optionally be combined with the subject matter of one or any combination of Aspects 1-3 to optionally include the articular surface is a convex surface.

Aspect 5 can include, or can optionally be combined with the subject matter of one or any combination of Aspects 1-4 to optionally include the tray member comprises a stem extending from a proximal portion of the tray member.

Aspect 6 can include, or can optionally be combined with the subject matter of one or any combination of Aspects 1-5 to optionally include a bearing disposed within the cavity where the articulation member includes a recess configured to mate with the bearing to allow movement of the articulation member within the cavity.

Aspect 7 can include, or can optionally be combined with the subject matter of one or any combination of Aspects 1-6 to optionally include a bearing extending within the cavity, wherein the bearing pivotably couples the tray member with the articulation member.

Aspect 8 can include, or can optionally be combined with the subject matter of one or any combination of Aspects 1-7 to optionally include the bearing is a ball bearing.

Aspect 9 can include, or can optionally be combined with the subject matter of one or any combination of Aspects 1-8 to optionally include the tray includes a cage on the base surface.

Aspect 10 can include, or can optionally be combined with the subject matter of one or any combination of Aspects 1-9 to optionally include the tray is a humeral tray.

Aspect 11 can include, or can optionally be combined with the subject matter of one or any combination of Aspects 1-10 to optionally include the articular surface is configured to receive a glenoid sphere.

Aspect 12 can include, or can optionally be combined with the subject matter of one or any combination of Aspects 1-11 to optionally include a bearing configured to pivotably couple the tray within the outer casing.

Aspect 13 can include an articulation implant comprising a static tray member configured to be coupled with a receiving bone, the static tray member including a first side configured to face the receiving bone, a second side opposite to the first side, the second side including a cavity opening, an inner wall of the tray member defining a cavity and accessible through the cavity opening; an articulation member configured to be at least partially retained within the cavity, the articulation member including an articular surface configured to face away from the cavity and a base surface configured to face toward the cavity; and a bearing configured to allow movement of the articulation member relative to the static tray member.

Aspect 14 can include, or can optionally be combined with the subject matter of Aspect 13, to optionally include a rim extending at least partially along an opening of the cavity; wherein the rim is configured to inhibit the articulation member from translating outside of the cavity.

Aspect 15 can include, or can optionally be combined with the subject matter of one or any combination of Aspects 13 or 14 to optionally include the bearing is retained within the cavity.

Aspect 16 can include, or can optionally be combined with the subject matter of one or any combination of Aspects 13-15 to optionally include wherein the bearing is a ball bearing.

Aspect 17 can include, or can optionally be combined with the subject matter of one or any combination of Aspects 13-17 to optionally include a shaft is configured to extend and support the bearing.

Aspect 18 can include, or can optionally be combined with the subject matter of one or any combination of Aspects 13-17 to optionally include the articulation member is configured to pivot about the bearing.

Aspect 19 can include, or can optionally be combined with the subject matter of one or any combination of Aspects 13-18 to optionally include the bearing pivotably couples the articulation member with the static tray member.

Aspect 20 can include, or can optionally be combined with the subject matter of one or any combination of Aspects 13-19 to optionally include the articular surface is concave.

Aspect 21 can include, or can optionally be combined with the subject matter of one or any combination of Aspects 13-20 to optionally include the articular surface is convex.

Aspect 22 can include, or can optionally be combined with the subject matter of one or any combination of Aspects 13-21 to optionally include the articular surface is configured to receive a glenoid sphere.

Aspect 23 can include, or can optionally be combined with the subject matter of one or any combination of Aspects 13-22 to optionally include the tray member includes a stem extending from the second side.

Aspect 24 can include, or can optionally be combined with the subject matter of one or any combination of Aspects 13-23 to optionally include the static tray member is coupled with either the humerus or the scapula.

Aspect 25 can include, or can optionally be combined with the subject matter of one or any combination of Aspects 13-24 to optionally include the articulation member is a socket configured to receive a rounded implant and wherein the articulation member is configured to articulate according to forces applied by the rounded implant.

Each of these non-limiting aspects can stand on its own, or can be combined in various permutations or combinations with one or more of the other aspects.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel,” “perpendicular,” “round,” or “square,” are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples, or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An implantable device comprising:
a tray member configured to be attached to a receiving bone, the tray member including:
a first side configured to face the receiving bone;
a second side opposite to the first side and including a cavity opening; and
a cavity defined within the tray member and accessible through the cavity opening; and
an articulation member configured to be at least partially retained within the cavity, the articulation member including:
an articular surface configured to face away from the cavity; and
a base surface configured to face towards the cavity;
wherein the articulation member is configured to be operably coupled to the tray member such that the articulation member can pivot within the cavity.

2. The implantable device of claim 1, including a liner coupled to the articular surface;
wherein the liner comprises a concave surface or a convex surface.

3. The implantable device of claim 1, wherein the articular surface includes a concave surface or a convex surface.

4. The implantable device of claim 1, wherein the tray member includes a stem extending from a proximal portion of the tray member.

5. The implantable device of claim 1, including a bearing disposed within the cavity;
wherein the articulation member includes a recess configured to mate with the bearing to allow movement of the articulation member within the cavity.

6. The implantable device of claim 1, including a bearing extending within the cavity;
wherein the bearing pivotably couples the tray member with the articulation member.

7. The implantable device of claim 6, wherein the bearing is a ball bearing.

8. The implantable device of claim 1, wherein the tray member includes a cage on the base surface.

9. The implantable device of claim 1, wherein the tray member is a humeral tray.

10. The implantable device of claim 1, wherein the articular surface is configured to receive a glenoid sphere.

11. An articulation implant comprising:
a static tray member configured to be coupled with a receiving bone, the static tray member including:
a first side configured to face the receiving bone;
a second side opposite to the first side, the second side including a cavity opening; and
an inner wall of the static tray member defining a cavity and accessible through the cavity opening;
an articulation member configured to be at least partially retained within the cavity, the articulation member including:
an articular surface configured to face away from the cavity; and
a base surface configured to face toward the cavity; and
a bearing configured to allow movement of the articulation member relative to the static tray member.

12. The articulation implant of claim 11, including a rim extending at least partially along an opening of the cavity;
wherein the rim is configured to inhibit the articulation member from translating outside of the cavity.

13. The articulation implant of claim 11, wherein the bearing is retained within the cavity.

14. The articulation implant of claim 11, wherein the bearing is a ball bearing.

15. The articulation implant of claim 11, wherein the articulation member is configured to pivot about the bearing.

16. The articulation implant of claim 11, wherein the bearing pivotably couples the articulation member with the static tray member.

17. The articulation implant of claim 11, wherein the articular surface is concave or convex.

18. The articulation implant of claim 11, wherein the articular surface is configured to receive a glenoid sphere.

19. The articulation implant of claim 11, wherein the static tray member is coupled with either a humerus or a scapula.

20. The articulation implant of claim 11, wherein the articulation member is a socket configured to receive a rounded implant; and
wherein the articulation member is configured to articulate according to forces applied by the rounded implant.
